(19) United States
(12) Patent Application Publication
(10) Pub. No.: US 2006/0051339 A1
(43) Pub. Date: Mar. 9, 2006

(54) Composition for treatment of oily skin and acne prevention and methods of making and using same

(76) Inventor: Hannah Naomi Sivak, Gilbert, AZ (US)

Correspondence Address: Hannah Naomi Sivak, 1213 W. Seascape Dr., Gilbert, AZ, 85233, US

(21) Appl. No.: 11/221,431

(22) Filed: Sep. 8, 2005

Related U.S. Application Data
(60) Provisional application No. 60/608,015, filed on Sep. 8, 2004.

(51) Int. Cl. A61K 38/44; A61K 38/46; A61K 31/355; A61K 31/375; A61K 36/484; A61K 36/61

(52) U.S. Cl. 424/94.6; 424/94.4; 514/458; 514/474; 514/763; 424/757

(57) ABSTRACT

Compositions and methods for the prevention and treatment of oily skin and the prevention and treatment of open comedos and acne using the enzyme lipase and an acceptable carrier are disclosed.

### RELATED APPLICATION DATA

This application claims the benefit of U.S. Provisional Application No. 60/608,015, filed on Sep. 8, 2004.

### BACKGROUND OF THE INVENTION

The skin sebaceous glands secrete sebum whose function is probably to enhance the skin's role as a barrier to external agents. Hormonal changes during puberty often trigger excess production of sebum.

Excess sebum secretion results in esthetically unpleasant shiny skin and comedos that often lead to acne. The initial acne lesion is the comedo, which occurs when a pore is clogged with a “plug” consisting of keratinocytes and excess sebum. A comedo can be open (blackhead) or closed (whitehead). These clogged pores are a perfect growing medium for bacteria such as Propionibacterium acnes, and infection by this bacterium will result in inflammation and a variety of acne lesions (papules, pustules, etc.).

Present methods to remove excess of superficial sebum include the use of absorbent paper, detergents and solvents like ethanol. Although detergents and alcohol take care of the immediate problem, these agents tend to irritate the skin and increase sebum production in the long term.

Part of a regime of acne prevention includes the frequent removal of pore obstruction, a task that can be made difficult and painful by the hard consistency of the lipid and keratinocyte plug. Comedo removal is done manually with or without the aid of a metal tool. A few years ago, nose strips were introduced that are applied to wet skin and, when dried, remove lipid-keratinocyte plugs that stick to them. This method is fast and painless but very inefficient, because it removes only a small proportion of the plugs, leaving behind many more.

Lipases are enzyme that hydrolyze ester bonds in triglycerides, resulting in the formation of free fatty acids, diglycerides, monoglycerides, and glycerol. The lipase enzyme is a hydrolase identified by the Enzyme Commission classification number 3.1.1.3.

Lipases are present in the pancreatic and intestinal juice of vertebrates, in plants, bacteria and fungi, etc.

Food grade lipases are readily available: pancreatic and fungal lipases are used in the food industry in the manufacture of cheese, and as food supplements for people who have digestive problems. Lipases from different organisms have been affirmed by the Food and Drug Administration to be GRAS (generally recognized as safe) for use to hydrolyze fatty acid glycerides in different circumstances.

### SUMMARY OF THE INVENTION

The present invention provides a composition including lipase for the treatment of oily skin and prevention of acne. While the ways in which the invention addresses the various drawbacks of known compositions and methods of treating infections will be described in more detail below, in general, the present invention provides a composition including an active lipase preparation and a carrier configured to maintain the composition in contact with an affected area for a prolonged period of time.

### DESCRIPTION OF THE INVENTION

The invention relates to methods of managing excess sebum production and/or open comedos which involve administering to a patient a therapeutically effective amount of a dermatological agent including the enzyme lipase in a pharmacologically acceptable carrier. The dermatological agent will be applied topically.

The dermatological agent may be administered with one or more additional pharmaceutical compositions to facilitate managing the dermatological condition. As used herein, the term “lipase” encompasses lipases natural or transgenic, and obtained from animal, plants, fungi and/or bacteria.

Many embodiments incorporate at least one other active ingredient with the lipase. The additional pharmaceutical composition may be an antibacterial, anti-inflammatories, antioxidants, vitamins, etc.

In the preferred practice of the invention, lipase is applied in admixture with a dermatologically acceptable carrier or vehicle (e.g., as a gel, lotion, cream or serum) so as to facilitate topical application and, in some cases, provide additional therapeutic effects as might be brought about by moisturizing the affected skin areas. As noted, other ingredients, are advantageously included in the compositions.

The amount of lipase necessary to bring about prevention and/or treatment is not fixed, and is dependent upon the source and purity of the enzyme, the amount and type of any additional ingredients used, particularly those that appear to exhibit synergistic effects, the skin type of the user, and, where present, the severity and extent of skin affected. Generally, the lipase or composition containing it is topically applied in effective amounts to skin or mucosa areas which are affected, or have been affected, by excess sebum production, comedos and/or acne.

In one embodiment, the composition contains from about 0.1% to about 10% (volume per volume), preferably from more than 0.1% or 0.2% to about 1% lipase.

While the carrier for lipase can be very simple, such as saline or phosphate buffer, it is generally preferred that the carrier be a composition that will facilitate topical application, and particularly one which will form a film or layer on the skin to which it is applied so as to localize the active ingredient. Many such compositions are known in the art, and can take the form of gels, lotions, creams, serums, etc. Typical compositions include thickeners, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a gel, or into solid sticks by utilization of different proportions of the ingredients and/or by inclusion of thickening agents such as gums or other forms of hydrophilic colloids. Such compositions are referred to herein as dermatologically-acceptable carriers.

One such embodiment contains as follows: 0.4% Xanthan gum, 4% Glycerin, 0.5% lipase powder, 95% Aloe gel Two volunteers applied this gel to their faces twice a day for a two weeks and reported that their skin was less oily and that no new acne lesions appeared. Many preferred embodiments of this invention contain at least one or two, and sometimes several, other active ingredients in addition to lipase, provided that the ingredients are compatible with lipase.

Liquorice extract may also be added to the lipase composition, alone or in combination with epigallocatechin gallate (EGCG) from green tea, in some embodiments.

The method of the present invention is useful to prepare a lotion or gel to pre-treat the skin in areas with abundance of comedos before the use of comedo removal nose strips or the manual removal of comedos by a professional. Same volunteers as above reported that the number of comedos removed using nose strips was higher when the area affected was pre-treated with the gel.

The method of the present invention is particularly useful for the prevention of recurrence of acne, alone or with other active ingredients can thus be added to dermatological creams and emollients as well as to commercial sunscreens, as the presence of comedos is often followed by infection by Propionibacterium acnes.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

I claim:

1. A method for the treatment and prevention of oily and of comedo and acne prone skin, the method comprising the step of applying a composition containing lipase and a carrier comprising a thickening agent to the affected area.

2. The method of claim 1, wherein the step of applying further comprises providing a carrier including liquorice extract.

3. The method of claim 1, wherein the step of applying further comprises providing an ingredient selected from the group consisting epigallocatechin gallate, propolis extract, tea tree oil, niacinamide.

4. The composition of claim 1, further comprising an antimicrobial agent.

5. The composition of claim 4, wherein the antimicrobial agent is selected from the group consisting of bee propolis and tea tree oil.

2. The method of claim 1, wherein the step of applying further comprises providing an ingredient selected from the group consisting of tocotrienols, vitamin E, ascorbic acid, superoxide dismutase, catalase, astaxanthin, lycopene.

5. A composition for treatment of oily skin and skin prone to comedos and acne, the composition comprising lipase and an a carrier comprising a moisturizing agent.

6. A composition for the treatment of oily skin and skin prone to comedos and acne, the composition comprising: lipase; and a carrier configured to maintain the active agent in contact with a skin of a patient for an extended period of time.
